(19) United States
(12) Patent Application Publication
(10) Pub. No.: US 2021/0205364 A1
(43) Pub. Date: Jul. 8, 2021

(54) LONG LIVED ENGINEERED T CELLS FOR ADOPTIVE CELL THERAPY

(71) Applicant: CASE WESTERN RESRVE UNIVERSITY, Cleveland, OH (US)

(72) Inventors: Rafick Pierre Sekaly, Cleveland, OH (US); Susan Pereira Robeiro, Cleveland, OH (US); Ashish Arunkumar Sharma, Cleveland, OH (US); Filipa Blasco Tavares Pereira Lopes, Cleveland, OH (US); Joumana Zeidan, Cleveland, OH (US)

(21) Appl. No.: 17/099,402

(22) Filed: Nov. 16, 2020

Related U.S. Application Data
(63) Continuation-in-part of application No. PCT/US2019/032426, filed on May 15, 2019.
(60) Provisional application No. 62/671,741, filed on May 15, 2018.

(51) Int. Cl. A61K 35/17; C07K 16/28

(52) U.S. Cl. CPC A61K 35/17; C07K 16/2809; C07K 16/2818

(57) ABSTRACT

A method of generating an enriched population of T cells for use in adoptive immunotherapy applications includes isolating T-cells from a biological sample of a subject, and separating a population of CD4/CD8 T cells having a CD45RA^{int}CD45RO^{int} phenotype from the isolated T cells.

### RELATED APPLICATION

This application is a Continuation-in-Part of PCT/US2019/32426, filed May 15, 2019, which claims priority from U.S. Provisional Application No. 62/671,741, filed May 15, 2018, the subject matter of which is incorporated herein by reference in its entirety.

### BACKGROUND

Cancer is one of the deadliest threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult. One of the difficulties in modern cancer treatments is the amount of time that elapses between a biopsy and the diagnosis of cancer, and effective treatment of the patient. During this time, a patient's tumor may grow unimpeded, such that the disease has progressed further before treatment is applied. This negatively affects the prognosis and outcome of the cancer.

Various strategies have been developed for producing and administering engineered cells for adoptive cell therapy (ACT). Adoptive transfer of tumor-infiltrating lymphocytes (TILs) has been successful in the treatment of patients with cancer. However, adoptive immunotherapy with tumor-reactive T cells derived from TIL has been used almost exclusively to treat patients with malignant melanoma because of the difficulty of isolating and expanding pre-existing tumor-reacting T cells from patients with tumor types other than melanoma. To overcome this limitation, patients' lymphocytes have been genetically engineered to express tumor antigen-specific receptors. For example, strategies are available for producing T cells expressing genetically engineered antigen receptors, such as T cell receptors (TCRs) and Chimeric antigen receptors (CARs). TCR T cells are autologous T cells that have been genetically engineered to express tumor antigen-specific TCRs that include α and β chains of TCR genes derived from a tumor-reactive allogenic T-cell clone. CAR T cell immunotherapy has emerged as a promising therapy for cancer. CAR T cells are autologous cells, engineered with an anti-tumor construct, that are effective at killing tumor cells. CARs are hybrid molecules comprising three essential units: (1) an extracellular antigen-binding motif, (2) linking/transmembrane motifs, and (3) intracellular T-cell signaling motifs. The antigen-binding motif of a CAR is commonly fashioned after a single chain Fragment variable (scFv), the minimal binding domain of an immunoglobulin (Ig) molecule. Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or γδ-T cells) are also under development.

Subject treated with TCR T cells engineered to express a modified high-avidity TCR specific for NY-ESO-1 antigen demonstrated objective clinical responses in 60% of patients with synovial cell sarcomas and 45% of patients with melanoma. As many as 70% of subjects treated with CAR T cells show complete clinical responses in blood cancer trials. Diverse tumor microenvironments, patient-to-patient variability and durability of therapy have all contributed to the effectiveness of CAR T and TCR T therapy. More importantly, the heterogeneity of engineered T cells produced using current protocols has contributed to the variable efficacy of this therapy. These protocols can enrich for short-lived differentiated cells, which are incapable of providing long-lived anti-tumor responses. To address this issue, a large number of engineered T cells are infused into the patient, making the therapy expensive. There remains significant work with regard to defining the most active T cell population to transduce with engineered antigen receptor vectors, determining the optimal culture and expansion techniques, and defining the molecular details of the engineered antigen receptor protein structure itself.

### SUMMARY

Embodiments described herein relate to a long-lived enriched population of CD4 T cells and CD 8 T cells (CD4/CD8 T cells) having a CD45RA^{int}CD45RO^{int} phenotype and to their use in compositions for T cell adoptive immunotherapy and treating cancer or an infectious disease in a subject in need thereof. It was found that a subset CD4/CD8 T cells has phenotypic and molecular attributes of long-lived pluripotent stem cells. Like other known stem cell populations, this subset population has a low metabolic profile (upregulation of fatty acid metabolism and oxidative phosphorylation, and down regulation of cell cycling pathways) retains the capacity to self-renew and can differentiate to effector cells. This subset is primarily characterized by intermediate co-expression of CD45RA and CD45RO (CD45RA^{int}CD45RO^{int}). CD4/CD8 T cells having a CD45RA^{int}CD45RO^{int} phenotype can also express CD95 (Fas) CD127 (IL7R) and CD27. Addition of low doses of cytokines IL-7 and IL-15 can lead to the formation of an enriched population of CD4/CD8 cells having the CD45RA^{int}CD45RO^{int} phenotype; while high doses of cytokines IL-7 and IL-15 can lead to effector differentiation of the cells.

CD4/CD8 T cells having a CD45RA^{int}CD45RO^{int} phenotype can be genetically modified to express one or more antigen-specific receptors in the T cells for use in adoptive immunotherapy applications to treat cancer or an infectious disease in a subject in need thereof. Advantageously, quantitatively lower amounts (e.g., 10 to 1000 fold lower amounts) of genetically modified CD4/CD8 T cells having a CD45RA^{int}CD45RO^{int} phenotype compared to conventional T cells can be infused into patients to generate a robust long-lasting anti-cancer or anti-tumor response, which results in cancer or tumor reduction, elimination, and/or remission. The antigen-specific receptors in the T cells can be selected from T cell receptors (TCRs) and chimeric antigen receptors (CARs).

In some embodiments, a method of generating an enriched population of CD4/CD8 T cells having a CD45RA^{int}CD45RO^{int} phenotype, which can optionally be genetically modified to express antigen-specific receptors includes isolating T-cells from a biological sample of a subject. The biological sample can include a T cell containing sample, such as peripheral blood mononuclear cells, of a subject having cancer to be treated, i.e., autologous T-cells from the subject to be treated. The isolated T cells can include CD4+ T cells and/or CD8+ T cells

In some embodiments, the isolated T cells can be genetically modified to express single or multiple antigen-specific receptors, which can recognize a cancer or infectious disease related antigen. In some embodiments, the CD4/CD8 T cells having a CD45RA^{int}CD45RO^{int} phenotype can then be separated from the genetically modified isolated CD4/CD8 T cells.

In some embodiments, the isolated CD4/CD8 T-cells are genetically modified by at least one of transduction, transfection, and/or electroporation to express the single or multiple antigen-specific receptors. The antigen-specific receptors can include an extracellular antigen binding domain that targets a cancer related antigen, such as CD19, CD20, CD22, ROR1, TSLPR, mesothelin, CD33, CD38, CD123 (IL3RA), CD138, BCMA (CD269), GPC2, GPC3, FGFR4, c-Met, PSMA, Glycolipid F77, EGFRvIII, GD-2, NY-ESO-1 TCR, MAGE A3 TCR, or combinations thereof.

In some embodiments, the separated CD4/CD8 T cells can express at least one of CD95, CD127, or CD27. In other embodiments, the CD4/CD8 T cells can intermediately express 4-1BB and optionally express OX40.

In other embodiments, the separated CD4/CD8 T-cells can express at least one of, at least two of, at least three of, at least four of, at least five of or more of IL17RA, CD5, IL2RG, IGF2R, SLC38A1, IL7R, SLC44A2, SLC2A3, CD96, CD44, CD6, CCR2b, CCR4, IL4R, or SLC12A7.

In some embodiments, the separated CD4/CD8 T-cells can have a CD45RA^{int}CD45RO^{int}CD95+CD127+CD27+phenotype. In other embodiments, the separated CD4/CD8 T-cells can have a CD45RA^{int}CD45RO^{int}CD95+CD127+CD27+IL7R+CD44+SCL38A1+IL2RG+CD6+CD5+phenotype.

In other embodiments, the method can include activating the isolated CD4/CD8 T cells with an anti-CD3 antibody and/or an anti-CD28 antibody prior to genetic modification and/or separation. The activated CD4/CD8 T cells can be cultured in an amount of IL7 and IL15 effective to promote expansion and/or formation of an enriched population of CD4/CD8 T cells having a CD45RA^{int}CD45RO^{int} phenotype. Once separated, the CD4/CD8 T cells having a CD45RA^{int}CD45RO^{int} phenotype can be cultured in a culture medium comprising TGFβ/IL1β to maintain the CD45RA^{int}CD45RO^{int} phenotype.

Other embodiments, described herein relate to adoptive immunotherapy composition that includes an enriched population of genetically modified CD4/CD8 T cells produced by a method described herein. At least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 75%, at least about 80%, at least 85%, at least about 90%, at least about 95% of the enriched population of genetically modified CD4/CD8 T cells can have a CD45RA^{int}CD45RO^{int} phenotype. The adoptive immunotherapy composition or enriched T-cell population can be administered to a subject with cancer or an infectious disease to treat the subject in need thereof. In some embodiments, administration of the adoptive immunotherapy composition or enriched T-cell population to a subject with cancer is capable of promoting in vivo expansion, persistence of patient specific anti-cancer T-cells resulting in cancer reduction, elimination, and/or remission.

In some embodiments, the cancer treated with adoptive immunotherapy composition can be a hematological cancer, such as leukemia, lymphoma, or multiple myeloma. The leukemia can be a chronic lymphocytic leukemia (CLL), acute lymphocytic leukemia (ALL), acute myeloid leukemia (AML), or chronic myelogenous leukemia (CIVIL). The lymphoma is mantle cell lymphoma, non-Hodgkin's lymphoma or Hodgkin's lymphoma.

In other embodiments, the cancer treated with adoptive immunotherapy composition can be an adult carcinoma comprising oral and pharynx cancer (tongue, mouth, pharynx, head and neck), digestive system cancers (esophagus, stomach, small intestine, colon, rectum, anus, liver, intrahepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

### BRIEF DESCRIPTION OF THE DRAWINGS

FIG. 1 is a flow diagram illustrating a method of generating an enriched population of CD4/CD8 T cells having a CD45RA^{int}CD45RO^{int} phenotype.

FIG. 2 illustrates expanded CAR T cells have a long-lived pluripotent stem T cell like phenotype. Expanded CAR+CD4 T cells when contrasted against fresh CD4 T cells, show intermediate expression of CD45RA and CD45RO. This population can be further identified as expressing CD95, CD127 and CD27.

FIG. 3 illustrates expanded CAR T cells show low glycolytic and effector machinery. These cells do not express master transcription factors of T cell differentiation (GATA3 and T-bet), and lack the expression of glycolytic enzymes like GLUT1, HK2 and PKM2. The effector phenotype can be rescued after stimulation with IL-15 for 48 hours.

FIG. 4 illustrates cells expressing RA^{int}RO^{int} phenotype have a quiescent gene expression profile. When compared to central and effector memory subsets, the RA^{int}RO^{int} cell subset had lower expression of cell cycling pathways and higher expression of fatty acid metabolism (associated with senescence).

FIG. 5 illustrates the stimulation of expanded CAR T cells for 48 hours in the presence of IL-15 causes them to upregulate p-STAT5, a downstream target of IL-15 signaling; and results in an increase in the proportion of CD27-cells, associated with an increased effector phenotype.

FIG. 6 illustrates protein levels of glycolytic enzymes, like PKM2, are lower in expanded CAR T cells and are maintained at a low level following stimulation with IL-1b and TGF-b (sustainers of the stem cell phenotype).

FIG. 7 illustrates graphs comparing the % of CD4/CD8 T-cells having a CD45RA^{int}CD45RO^{int} phenotype cultured in low IL-7/IL-15 conditions compared to high IL/IL15 conditions.

FIG. 8 illustrates plots showing high levels of IL-15 causes effector differentiation of of CD4/CD8 T cells having a CD45RA^{int}CD45RO^{int} phenotype and stem cell fate of CD4/CD8 T cells having a CD45RA^{int}CD45RO^{int} phenotype is maintained with TGFβ/I1β administration.

### DETAILED DESCRIPTION

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1988; Houston et al., 1988; Bird et al., 1988).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of cells of the invention in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, melanoma, lung cancer and the like.

The term “infectious disease” as used herein is defined as a disorder caused by pathogenic microorganisms, such as bacteria, viruses, parasites or fungi that are typically spread directly or indirectly (vector-borne) from one individual to another.

The term “T cell receptor” or alternatively a “TCR” refers to a group of polypeptide chains (a or β) found on T lymphocyte cells that recognize and bind to certain antigens (proteins) found on abnormal cells, cancer cells, cells from other organisms, and cells infected with a virus or another microorganism. TCRs are antigen specific; their activity depends on antigen processing by macrophages or other antigen presenting cells and the presence of major histocompatibility complex proteins to which peptides from the antigen are bound.

The term “Chimeric Antigen Receptor” or alternatively a “CAR” refers to a set of polypeptides, typically two in the simplest embodiments, which when in a T cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below.

In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule.

The term “signaling domain” refers to the functional portion of a protein which acts by transmitting information within the cell to regulate cellular activity via defined signaling pathways by generating second messengers or functioning as effectors by responding to such messengers.

The term “intracellular signaling domain,” as the term is used herein, refers to an intracellular portion of a molecule. The intracellular signaling domain can generate a signal that promotes an immune effector function of the CAR containing cell, e.g., a CAR T cell. Examples of immune effector function, e.g., in a CAR T cell, include cytolytic activity and helper activity, including the secretion of cytokines. In embodiments, the intracellular signaling domain is the portion of a protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR T, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3ζ, FcRγ, FcRβ, CD3γ, CD3δ, CD3ε, CD5, CD22, CD79a, CD79b, CD278 (“ICOS”), CD66d, CD32, DAP10, and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

The intracellular signaling domain can comprise the entire intracellular portion, or the entire native intracellular signaling domain, of the molecule from which it is derived, or a functional fragment thereof.

The term “effector function” refers to a specialized function of a cell. Effector function of a T cell, for example, may be cytolytic activity or helper activity including the secretion of cytokines.

The terms “cancer associated antigen” or “tumor antigen” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In certain aspects, the tumor antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell.

The term “infectious disease antigen” as used herein is an antigen associated with or expressed, e.g., specifically expressed, on or in an infectious disease or condition or cell or pathogenic microorganism, such as bacteria, viruses, parasites or fungi to be treated.

An “effective amount” as used herein, means an amount which provides a therapeutic or prophylactic benefit.

The term “expression” as used herein is defined as the transcription and/or translation of a particular nucleotide sequence driven by its promoter.

The term “specifically binds,” as used herein, is meant a molecule, such as an antibody, which recognizes and binds to another molecule or feature, but does not substantially recognize or bind other molecules or features in a sample.

The term “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Embodiments described herein relate to a long-lived or persistent enriched population of genetically engineered CD4 T cells and CD 8 T cells (CD4/CD8 T cells) having a CD45RA^{int}CD45RO^{int} phenotype and to their use in compositions for T cell adoptive immunotherapy and treating cancer or an infectious disease in a subject in need thereof. It was found that a subset CD4/CD8 T cells has phenotypic and molecular attributes of long-lived pluripotent stem cells. Like other known stem cell populations, this subset population has a low metabolic profile (upregulation of fatty acid metabolism and oxidative phosphorylation, and down regulation of cell cycling pathways) retains the capacity to self-renew, and can differentiate to effector cells. This subset is primarily characterized by intermediate co-expression of CD45RA and CD45RO (CD45RA^{int}CD45RO^{int}). CD4/CD8 T cells having a CD45RA^{int}CD45RO^{int} phenotype can also express CD95 (Fas) CD127 (IL7R) and CD27. Addition of low doses of cytokines IL-7 and IL-15 can induce the formation of an enriched population of CD4/CD8 cells having the CD45RA^{int}CD45RO^{int} phenotype; while high doses of cytokines IL-7 and IL-15 can lead to effector differentiation of the cells.

The enriched population CD4/CD8 T cells having a CD45RA^{int}CD45RO^{int} phenotype upon transplantation or administration to a subject have the ability to persist or survive long term in the subject. The persistence can correlate with the efficacy of a therapeutic T cell transplant in the treatment of a disease, such as cancer or an infectious disease. The greater the persistence of therapeutic T cells, the more likely a therapeutic regime is to be effective, for example the less likely a tumor or infection relapse will occur. Thus, long-lived, self-renewing and pluripotent CD4/CD8 T cells having a CD45RA^{int}CD45RO^{int} phenotype can have a reduced cost of production, promote effector differentiation, and increase efficiency of genetically engineered T cell adoptive immunotherapy. Moreover, frequencies of these cells in the current available, T cell therapy products, such as CAR T cell therapy products, can be used as a biomarker, and predictive of successful intervention.

In some embodiment, the enriched population CD4/CD8 T cells having a CD45RA^{int}CD45RO^{int} phenotype can persist in vivo for at least 1, 2, 3, 4, 5, 6, 12, 24, 36, 48 or 72 months longer than T cells without the CD45RA^{int}CD45RO^{int} phenotype after administration to a subject. The enriched population CD4/CD8 T cells having a CD45RA^{int}CD45RO^{int} phenotype can also possess an increased ability to engraft in a subject after administration. In particular, the enriched population CD4/CD8 T cells having a CD45RA^{int}CD45RO^{int} phenotype can possess an increased ability to engraft in a non-conditioned recipient (e.g., a recipient who has not undergone chemotherapy and/or radiotherapy conditioning).

The term “engraftment” refers to the ability of the transplanted cells to populate a recipient and survive in the immediate aftermath of their transplantation. Accordingly, engraftment is assessed in the short term after transplantation. For example, engraftment may refer to the number of cells descended from the transplanted cells that are detected in the first in vivo evaluation of an experiment, clinical trial or therapeutic protocol, e.g., at the earliest time point that transplanted cells or their descendants may be detected in a recipient. In one embodiment, engraftment is assessed at 0-12, 0-24, 0-48 or 0-72 h after transplantation. In another embodiment, engraftment is assessed at about 1, 2, 3, 4, 5, 6, 12, 24, 36, 48, 60 or 72 h after transplantation. In a preferred embodiment, engraftment is assessed at about 12 h after transplantation.

CD4/CD8 T cells having a CD45RA^{int}CD45RO^{int} phenotype can be genetically modified to express antigen-specific receptors in the T cells and be used in adoptive immunotherapy applications to treat cancer or an infectious disease in a subject in need thereof. In certain embodiments, the antigen-specific receptors in the T cells can be selected from T cell receptors (TCRs) and chimeric antigen receptors (CARs). Advantageously, quantitatively lower amounts (e.g., 10 to 1000 fold lower amounts) of CD4/CD8 T cells that are genetically modified to express antigen-specific receptors and having a CD45RA^{int}CD45RO^{int} phenotype compared to conventional T cells can be infused into patients to generate a robust long-lasting anti-cancer, anti-tumor, or anti-microbial response, which results in cancer, tumor or infection reduction, elimination, and/or remission.

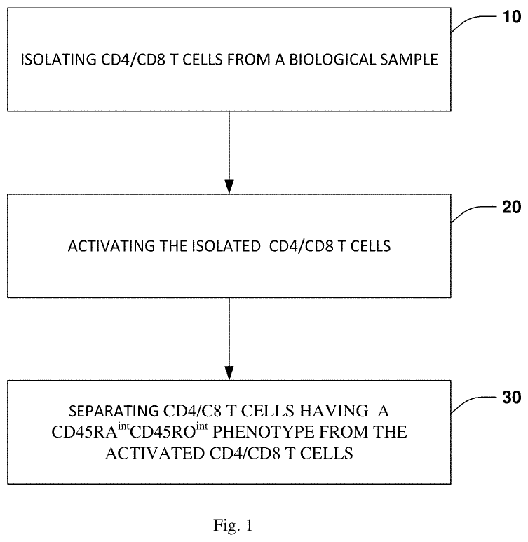

FIG. 1 illustrates a flow diagram illustrating a method of generating an enriched population of CD4/CD8 T cells having a CD45RA^{int}CD45RO^{int} phenotype, which can be genetically modified to express one or more antigen-specific receptors. In the method, at step 10, a naïve population of T-cells is isolated from a biological sample of a subject. The biological sample can include any T cell containing sample from the subject. Examples of subjects include humans, dogs, cats, mice, rats, and transgenic species thereof. Preferably, the subject is a human.

T cells can be obtained from a number of sources, including peripheral blood mononuclear cells, bone marrow, lymph node tissue, spleen tissue, and tumors. In some embodiments, the T cells can be obtained from a subject having cancer or an infectious disease to be treated, i.e., autologous T-cells from the subject to be treated. In certain embodiments, T cells can be obtained from a unit of blood collected from a subject using any number of techniques known to the skilled artisan, such as ficoll separation. In some embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. In one embodiment, the cells can be washed with phosphate buffered saline (PBS). In an alternative embodiment, the wash solution lacks calcium and may lack magnesium or may lack many or all divalent cations. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells can be isolated from peripheral blood by lysing the red blood cells and depleting the monocytes, for example, by centrifugation through a PERCOLL gradient. Alternatively, T cells can be isolated from umbilical cord. In any event, a specific subpopulation of T cells can be further isolated by positive or negative selection techniques.

In some embodiments, the isolated T cells can include CD4+ T cells and/or CD8+ T cells. CD4 T cells and/or CD8 T cells (CD4/CD8 T cells) can be isolated from the biological sample by positive or negative selection. Negative selection can be accomplished using a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

Following isolation of the T cells from the biological sample, at step 20, the isolated CD4/CD8 T cells can be activated and/or expanded by any suitable method known in the art. In an embodiment of the invention, the T cells are activated and the numbers of T cells are expanded in the presence of one or more non-specific T cell stimuli (e.g., anti-CD3 and anti-CD28) and/or one or more cytokines, cytokines (e.g., IL-1b, IL-2, IL-4, IL-6, IL-7, IL-9, IL-10, IL-12, IL-15, IL-17, IL-21, IL-22, IL-23, IL-35, TGF-β, IFNα, IFNγ, TNFα) recombinant proteins, costimulatory molecules, lectins, ionophores, synthetic molecules, antigen presenting cells (APCs), artificial APCs or feeders. In some embodiments, the CD4/CD8 T cells can be activated and the numbers of T cells are expanded by physically contacting the T cells with one or more non-specific T cell stimuli and/or one or more cytokines. Any one or more non-specific T cell stimuli may be used in the inventive methods. Examples of non-specific T cell stimuli include anti-CD3 antibodies and anti-CD28 antibodies. In some embodiments, the non-specific T cell stimulus may be anti-CD3 antibodies and anti-CD28 antibodies conjugated to beads. Any one or more cytokines may be used in the inventive methods. Exemplary cytokines include interleukin (IL)-2, IL-7, IL-21, and IL-15.

Following activation and/or expansion of the isolated CD4/CD8 T cells, at step 30, the CD4/CD8 T cells can be separated or sorted using, for example, flow cytometry, into an enriched population of CD4/CD8 T cells characterized by intermediate co-expression of CD45RA and CD45RO (CD45RA^{int}CD45RO^{int}). The method may comprise sorting the cells in any suitable manner. In some embodiments, the sorting is carried out using flow cytometry. The flow cytometry may be carried out using any suitable method known in the art. The flow cytometry may employ any suitable antibodies and stains. In some embodiments, the flow cytometry is polychromatic flow cytometry.

The enriched population of CD4/C8 T cells having a CD45RA^{int}CD45RO^{int} phenotype produced by the processes described herein can include CD4/C8 T cells having a CD45RA^{int}CD45RO^{int} as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% CD4/C8 T cells having a CD45RA^{int}CD45RO^{int}. In preferred embodiments, the cells of the cell cultures or cell populations comprise human cells.

The long lived CD4/CD8 T cells having a CD45RA^{int}CD45RO^{int} phenotype also be characterized by the expression of other cell surface markers. For example, the separated CD4/CD8 T cells having a CD45RA^{int}CD45RO^{int} phenotype can express at least one of CD95, CD127, or CD27. In other embodiments, the CD4/CD8 T cells having a CD45RA^{int}CD45RO^{int} phenotype can further intermediately express 4-1BB and optionally express OX40.

In other embodiments, the separated CD4/CD8 T-cells having a CD45RA^{int}CD45RO^{int} phenotype can further express at least one of, at least two of, at least three of, at least four of, at least five of or more of IL17RA, CD5, IL2RG, IGF2R, SLC38A1, IL7R, SLC44A2, SLC2A3, CD96, CD44, CD6, CCR2b, CCR4, IL4R, or SLC12A7.

In some embodiments, the separated CD4/CD8 T cells can have a CD45RA^{int}CD45RO^{int}CD95+CD127+CD27+phenotype. In other embodiments, the separated CD4/CD8 T-cells can have a CD45RA^{int}CD45RO^{int l CD}95+CD127+CD27+IL7R+CD44+SCL38A1+IL2RG+CD6+CD5+phenotype.

In some embodiments, prior to and/or after separation or sorting of the CD4/C8 T cells having a CD45RA^{int}CD45RO^{int} phenotype, the isolated CD4-CD8 T cells having the CD45RA^{int}CD45RO^{int} phenotype can be enriched by culturing the isolated CD4/CD8 T cells in a culture medium that includes low amount of IL-7 and/or IL-15. As shown in FIG. 7, it was found that activated CD4/CD8 T cells cultured in low IL-7/IL-15 conditions (e.g., concentration of IL7/IL15 less than 10 ng/ml) can promote or form an enriched population of the CD4/C8 T cells having a CD45RA^{int}CD45RO^{int} phenotype compared to activated CD4/CD8 T cells cultured in high IL-7/IL-15 conditions (e.g., concentration of IL-7/IL-15 greater than 10 ng/ml).

In some embodiments, the culture medium can include IL-7 and/or IL-15 at a concentration, for example, of less than about 100 ng/ml, less than about 95 ng/ml, less than about 90 ng/ml, less than about 85 ng/ml, less than about 80 ng/ml, less than about 75 ng/ml, less than about 70 ng/ml, less than about 65 ng/ml, less than about 60 ng/ml, less than about 55 ng/ml, less than about 50 ng/ml, less than about 45 ng/ml, less than about 40 ng/ml, less than about 35 ng/ml, less than about 30 ng/ml, less than about 25 ng/ml, less than about 20 ng/ml, less than about 15 ng/ml, less than about 10 ng/ml, less than about 5 ng/ml, less than about 4 ng/ml, less than about 3 ng/ml, less than about 2 ng/ml, or less than about 1 ng/ml.

Using the low IL-7/IL-15 concentration culture medium described herein, cell populations or cell cultures can be enriched in CD4/C8 T cells having a CD45RA^{int}CD45RO^{int} phenotype content by at least about 2- to about 1000-fold as compared to untreated cell populations or cell cultures. In some embodiments, CD4/C8 T cells having a CD45RA^{int}CD45RO^{int} phenotype can be enriched by at least about 5- to about 500-fold as compared to untreated cell populations or cell cultures. In other embodiments, CD4/C8 T cells having a CD45RA^{int}CD45RO^{int} phenotype can be enriched from at least about 10- to about 200-fold as compared to untreated cell populations or cell cultures. In still other embodiments, CD4/C8 T cells having a CD45RA^{int}CD45RO^{int} phenotype can be enriched from at least about 20- to about 100-fold as compared to untreated cell populations or cell cultures. In yet other embodiments, CD4/C8 T cells having a CD45RA^{int}CD45RO^{int} phenotype can be enriched from at least about 40- to about 80-fold as compared to untreated cell populations or cell cultures. In certain embodiments, CD4/C8 T cells having a CD45RA^{int}CD45RO^{int} phenotype can be enriched from at least about 2- to about 20-fold as compared to untreated cell populations or cell cultures.

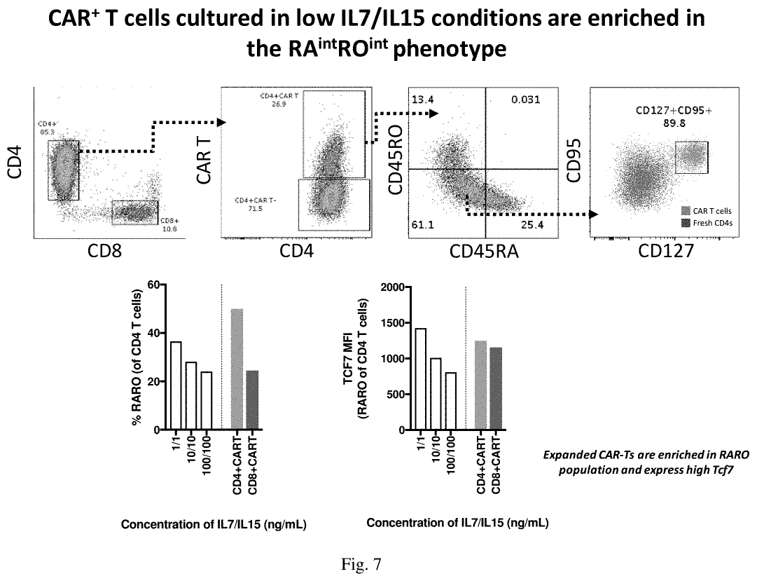

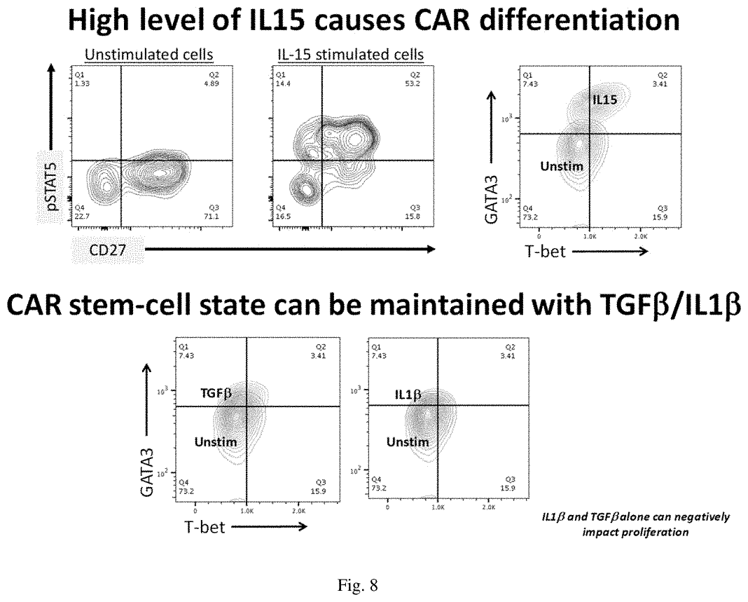

In some embodiments, once separated or sorted, the CD4/CD8 T-cells having a CD45RA^{int}CD45RO^{int} phenotype can be cultured in a culture medium comprising TGFβ/IL1β to maintain the CD45RA^{int}CD45RO^{int} phenotype. As illustrated in FIGS. 6 and 8, the addition of TGFβ and/or IL1β to the CD4/CD8 cells having a CD45RA^{int}CD45RO^{int} phenotype led to the maintenance of the CD45RA^{int}CD45RO^{int} phenotype prior to administration to a subject.

The method further includes genetically modifying the CD4/CD8 T cells prior to, or after, activation. In some embodiments, CD4/CD8 T cells are genetically modified with a nucleotide sequence encoding an antigen-specific receptor targeting (e.g., specifically binding to or recognizing) an antigen, such as a disease-specific target antigen corresponding to the disease or condition to be treated. In some embodiments, the CD4/CD8 T cells are modified to include one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the genetic modification of the CD4/CD8 T cells may be performed by transduction, transfection or electroporation. Transduction can performed with lentiviruses, gamma-, alpha-retroviruses or adenoviruses or with electroporation or transfection by nucleic acids (DNA, mRNA, miRNA, antagomirs, ODNs), proteins, site-specific nucleases (zinc finger nucleases, TALENs, CRISP/R), self-replicating RNA viruses (e.g., equine encephalopathy virus) or integration-deficient lentiviral vectors. For example, genetic modification of the CD4/CD8 T cells can be performed by transducing the CD4/CD8 T cells with lentiviral vectors

In some embodiments, the genetically engineered antigen receptor can include a T cell receptor (TCR) or components thereof, or a functional non-TCR antigen recognition receptor, such as chimeric antigen receptor (CAR), including chimeric activating receptors and chimeric costimulatory receptors. In some embodiments, the genetically engineered antigen receptor is capable of inducing an activating signal to the CD4/CD8 T cells. In some embodiments, the genetically engineered antigen receptor contains an extracellular antigen recognition domain which specifically binds to a target antigen at a dissociation constant (K_{D}) of at least 10⁻⁸M, at least 10⁻⁷M, at least 10⁻⁶M, at least 10⁻⁵M, or at least 10⁻⁴M.

In some embodiments, the genetically engineered antigen receptors include recombinant T cell receptors (TCRs) and/or TCRs cloned from naturally occurring T cells and/or pairs of chains of TCRs cloned from naturally occurring T cells. Exemplary antigen receptors, including CARs and recombinant TCRs, as well as methods for engineering and introducing the receptors into cells, include those described, for example, in international patent application publication numbers W0200014257, W02013126726, W02012/129514, W02014031687, W02013/166321, W02013/071154, W02013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the genetically engineered antigen receptors include a CAR as described in U.S. Pat. No. 7,446,190, and those described in International Patent Application Publication No.: W0/2014055668 A1.

In general, TCRs contain a variable α and β chain (also known as TCRα and TCRβ, respectively) or a variable γ and δ chain (also known as TCRγ and TCRδ, respectively) or antigen-binding portion(s) thereof, and in general are capable of specifically binding to an antigen peptide bound to a MHC receptor. Thus, TCR T cells can provide specificity and reactivity toward a selected target, but in an MHC-restricted manner.

In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). For example, in some aspects, each chain of the TCR can possess one N-terminal immunoglobulin variable domain, one immuno-globulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable α chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jares et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Va or V13; typically amino acids 1 to 116 based on Kabat numbering Kabat et al., “Sequences of Proteins of Immunological Interest, U.S. Dept. Health and Human Services, Public Health Service National Institutes of Health, 1991, 5th ed.) at the N-terminus, and one constant domain (e.g., α-chain constant domain or C_{α} typically amino acids 117 to 259 based on Kabat, β-chain constant domain or C_{β}, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ,CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, a TCR for a target antigen (e.g., a cancer antigen) is identified and introduced into the cells. In some embodiments, a TCR for a target antigen also specifically binds to, e.g., is cross-reactive with, one or more peptide epitopes of one or more other antigens, such as those that are related to (e.g., by way of sharing sequence or structural similarity with) the target antigen. The crossreactive antigen may have an epitope that is the same as or has one or more amino acid differences as compared to the target antigen, such as one, two, or three differences. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells, such as from a T cell (e.g. cytotoxic T cell), T-cell hybridomas or other publicly available source. In some embodiments, the T-cells can be obtained from in vivo isolated cells. In some embodiments, a T cell clone, such as a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof can be synthetically generated from knowledge of the sequence of the TCR.

In some embodiments, after the T-cell clone is obtained, the TCR α and β chains are isolated and cloned into a gene expression vector. In some embodiments, the TCR α and β genes are linked via a picomavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13: 1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, the method further includes genetically modifying the CD4/CD8 T cells prior to or after activation with a nucleotide sequence encoding a chimeric antigen receptor (CAR). The CAR may have antigenic specificity for a cancer antigen or an infectious disease antigen.

The CARs disclosed herein comprise at least one extracellular domain capable of binding to an antigen, at least one transmembrane domain, and at least one intracellular domain.

A chimeric antigen receptor (CAR) is an artificially constructed hybrid protein or polypeptide containing the antigen binding domains of an antibody (e.g., single chain variable fragment (scFv)) linked to T-cell signaling domains via a transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the intracellular T cell signaling domains of the CARs can include, for example, a T cell receptor signaling domain, a T cell costimulatory signaling domain, or both. The T cell receptor signaling domain refers to a portion of the CAR comprising the intracellular domain of a T cell receptor, such as, for example, and not by way of limitation, the intracellular portion of the CD3 zeta protein. The costimulatory signaling domain refers to a portion of the CAR comprising the intracellular domain of a costimulatory molecule, which is a cell surface molecule other than an antigen receptor or their ligands that are required for an efficient response of lymphocytes to antigen.

In some embodiments, the antigen-specific receptor used in the CD4/CD8 T-cell population(s) as disclosed herein, includes a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. In some embodiments, a target antigen that is expressed on or in, specifically expressed on or in , or associated with, the particular disease state or condition may be referred to as a “disease-specific target” “disease-specific antigen” or “disease-specific antigen”. Thus, examples of cell surface markers that may act as ligands for the antigen binding domain in the genetically engineered antigen-specific receptor include those associated with viral, bacterial and parasitic infections, autoimmune disease and cancer cells.

In one embodiment, the antigen-specific receptor can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens are well known in the art and include, for example, a glioma-associated antigen, carcinoembryonic antigen (CEA), CEACAM5, beta-human chorionic gonadotropin, αfetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, GD-2, prominin-1 (CD133), folate receptor alpha (FRa), and mesothelin. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20, CD22, and CD37 are other candidates for target antigens in B-cell lymphoma. Some of these antigens (CEA, HER-2, CD19, CD20, CD22, idiotype) have been used as targets for passive immunotherapy with monoclonal antibodies with limited success.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen binding domain portion of the antigen-specific receptor targets an antigen that includes but is not limited to CD19, CD20, CD22, ROR1, Mesothelin, CD33, c-Met, PSMA, Glycolipid F77, EGFRvIII, GD-2, MY-ESO-1 TCR, MAGE A3 TCR, and the like.

Depending on the desired antigen to be targeted, the antigen-specific receptor can be engineered to include the appropriate antigen bind domain that is specific to the desired antigen target. For example, if CD19 is the desired antigen that is to be targeted, an antibody for CD19 can be used as the antigen bind domain incorporation into the CAR.

In one exemplary embodiment, the antigen binding domain portion of the antigen-specific receptor is an antigen-specific receptor, such as a CAR, that targets CD19. Preferably, the antigen binding domain in the CAR is anti-CD19 scFV.

In another embodiment, scFvs can be replaced with a nanobody, such as a nanobody derived from camelids.

In other embodiments, an antigen-specific receptor can be expressed that is capable of binding to a non-TSA or non-TAA including, for example and not by way of limitation, an antigen derived from Retroviridae (e.g., human immunodeficiency viruses such as HIV-1 and HIV-LP), Picornaviridae (e.g., poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g., type 1 and type 2 herpes simplex virus (HSV), varicella-zoster virus, cytomegalovirus (CMV), and herpes virus], Poxviridae (e.g., smallpox virus, vaccinia virus, and pox virus), or hepatitis C virus, or any combination thereof.

In other embodiments, an antigen-specific receptor can be expressed that is capable of binding to an antigen derived from a bacterial strain of Staphylococci, Streptococcus, Escherichia coli, Pseudomonas, or Salmonella. Particularly, there is provided an antigen-specific receptor capable of binding to an antigen derived from an infectious bacterium, for example, Helicobacter pyloris, Legionella pneumophilia, a bacterial strain of Mycobacteria sps. (e.g., M. tuberculosis, M. avium, M. intracellulare, M. kansaii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani, or a combination thereof.

The one or more transmembrane domains fused to the extracellular domain of an antigen-specific receptor, such as CAR, can be derived either from a natural or from a synthetic source. Where the source is natural, the domain may be derived from any membrane-bound or transmembrane protein. Transmembrane regions of particular can be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain in the antigen-specific receptor, such as CAR, can be a CD8 transmembrane domain. Other non-limiting examples of transmembrane domains for use in the CARs disclosed herein include the TNFRSF16 and TNFRSF19 transmembrane domains may be used to derive the TNFRSF transmembrane domains and/or linker or spacer domains disclosed including, in particular, those other TNFRSF members listed within the tumor necrosis factor receptor superfamily.

In some embodiments, the CARs expressed in the CD4/CD8 T-cell population(s) as disclosed herein, include a spacer domain that can be arranged between the extracellular domain and the TNFRSF transmembrane domain, or between the intracellular domain and the TNFRSF transmembrane domain. The spacer domain means any oligopeptide or polypeptide that serves to link the TNFRSF transmembrane domain with the extracellular domain and/or the TNFRSF transmembrane domain with the intracellular domain. The spacer domain can include up to 300 amino acids, 10 to 100 amino acids, or 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,5667, 498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of an antigen-specific receptor, such as CAR, with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone can be insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARS disclosed herein include those derived from TCRζ (CD3ζ), FcRα, FcRβ, CD3γ, CD3δ, CD3ε, CD5, CD22, CD79a, CD79b, and CD66d. In one embodiment, the cytoplasmic signaling molecule in the CAR comprises a cytoplasmic signaling sequence derived from CD3 zeta. The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to comprise the signaling domain of CD3-ζ and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-ζ and the signaling domain of CD28 and 4-1BB.

Exemplary CARs include those described in International Patent Application Publication No. WO 2011041093 and International Application No. PCT/US 12/29861, each of which is incorporated herein by reference. Exemplary TCRs include those described in U.S. Pat. Nos. 7,820,174; 8,088,379; 8,216,565; U.S. Patent Application Publication No. 20090304657; and International Patent Application Publication Nos. WO 2012040012 and WO 2012054825, each of which is incorporated herein by reference. The cells may be transduced using any suitable method known in the art, for example, as described in Sambrook et al., Molecular Cloning: A Laboratory Manual, 3.sup.rd ed., Cold Spring Harbor Press, Cold Spring Harbor, N.Y. 2001; and Ausubel et al., Current Protocols in Molecular Biology, Greene Publishing Associates and John Wiley & Sons, N.Y., 1994.

In some embodiments, improved selectivity and specificity is achieved through strategies targeting multiple antigens. Such strategies generally involve multiple antigen binding domains, which typically are present on distinct genetically engineered antigen receptors and specifically bind to distinct antigens. Thus, in some embodiments, the CD4/CD8 T cells having the CD45RA^{int}CD45RO^{int} phenotype are engineered with the ability to bind more than one antigen. In some aspects, a plurality of genetically engineered antigen receptors are introduced into the cell, which specifically bind to different antigens, each expressed in or on the disease or condition to be targeted with the cells or tissues or cells thereof. Such features can in some aspects address or reduce the likelihood of off-target effects. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function.

In some embodiments, the CD4/CD8 T cells having the CD45RA^{int}CD45RO^{int} phenotype include other genetically engineered antigen-specific receptor, such as a costimulatory receptor, that specifically binds to another antigen and is capable of inducing a costimulatory signal to the cell. In some aspects, such another target antigen and the first target antigen recognized by the first antigen-specific receptor are distinct.

In some embodiments, the other genetically engineered antigen-specific receptor is one that is not expressed or is not specifically expressed or associated with the disease or condition. In some aspects the other genetically engineered antigen-specific receptor is one that may be expressed or associated with another cancer or infectious disease that is not targeted by the first target antigen, and in some aspects another antigen is not expressed or specifically expressed or associated with any cancer or infectious disease.

In some embodiments, ligation of the first genetically engineered antigen-specific receptor and the other engineered antigen-specific receptor (e.g., a second engineered antigen-specific receptor) induces a response in the CD4/CD8 T cell, which response is not induced by ligation of either of the genetically engineered antigen receptors alone. In some embodiments, the response is selected from the group consisting of proliferation, secretion or a cytokine, and cytotoxic activity.

In certain embodiments, CD4/CD8 T cells having the CD45RA^{int}CD45RO^{int} phenotype are further modified in order to increase their therapeutic or prophylactic efficacy. For example, in some embodiments, the engineered antigen-specific receptor expressed by the CD4/CD8 T cells having the CD45RA^{int}CD45RO^{int} phenotype can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

In some embodiments, CD4/CD8 T cells having the CD45RA^{int}CD45RO^{int} phenotype are further modified in order to enhance T cell trafficking/homing to targeted sites, such as tumor sites. For example, genetically engineered T cells expressing an antigen-specific receptor can be further modified with chemokine receptors that specifically bind chemokines produced by tumors. In some embodiments, genetically engineered T cells expressing an antigen-specific receptor can be further modified to coexpress CCR2 and/or CCR4. Genetically engineered T cells expressing VEGFR-1 have been shown to delay tumor growth and formation and suppress metastasis in tumor models. Therefore, in some embodiments, CD4/CD8 T cells having the CD45RA^{int}CD45RO^{int} phenotype are further modified to coexpress VEGFR-1.

Various immunosuppressive cytokines such as transforming growth factor (TGF)-β and IL-10, are involved in the inhibition of engineered T cell based cancer immunotherapy. In some embodiments, genetically engineered T cells expressing an antigen-specific receptor can express a dominant-negative TGF-β and/or IL-10 receptor. IL-2, IL-4, IL-7, IL-15, and IL-21 have been shown to mitigate the effects of immunosuppressive factors in the tumor microenvironment and enhance genetically engineered T cell efficacy. Therefore, CD4/CD8 T cells having the CD45RA^{int}CD45RO^{int} phenotype can be further genetically modified to express one or more of IL-2, IL-4, IL-7, IL-15, and IL-21.

Programmed cell death protein-1 (PD-1) has been implemented as a target to promote genetically engineered T cell efficacy. In some embodiments, CD4/CD8 T cells having the CD45RA^{int}CD45RO^{int} phenotype are further modified to genetically deplete PD-1. In some embodiments, CD4/CD8 T cells having the CD45RA^{int}CD45RO^{int} phenotype are further modified to coexpress PD-1 antibody.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

The enriched population of genetically engineered CD4/CD8 T cells having the CD45RA^{int}CD45RO^{int} phenotype can be included in a composition, such as a pharmaceutical composition, for immunotherapy, adoptive immunotherapy, and/or treating cancer or an infectious disease. The composition can also include a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use.

The compositions can be prepared in unit dosage forms for administration to a subject. The amount and timing of administration are at the discretion of the treating clinician to achieve the desired outcome. The compositions can be formulated for systemic (such as intravenous) or local (such as intra-tumor) administration. In one example, an enriched population of CD4/CD8 T cells having the CD45RA^{int}CD45RO^{int} phenotype genetically engineered to express an antigen-specific receptor is formulated for parenteral administration, such as intravenous administration. Compositions including an enriched population of genetically engineered CD4/CD8 T cells having the CD45RA^{int}CD45RO^{int} phenotype as disclosed herein can be used, for example, for the treatment a tumor.

The compositions for administration can include a solution of the enriched population of genetically engineered CD4/CD8 T cells having the CD45RA^{int}CD45RO^{int} phenotype provided in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions, such as pH adjusting and buffering agents, toxicity adjusting agents, adjuvant agents, and the like, for example, sodium acetate, sodium chloride, potassium chloride, calcium chloride, sodium lactate and the like. The concentration of the enriched population of genetically modified CD4/CD8 T cells having the CD45RA^{int}CD45RO^{int} phenotype in these formulations can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

In one example, the enriched population of genetically engineered CD4/CD8 T cells having the CD45RA^{int}CD45RO^{int} phenotype can be added to an infusion bag containing 0.9% sodium chloride, USP, and in some cases administered at a dosage of from 0.5 to 15 mg/kg of body weight. An enriched population of genetically engineered CD4/CD8 T cells having the CD45RA^{int}CD45RO^{int} phenotype can be administered by slow infusion, rather than in an intravenous push or bolus. In one example, a higher loading dose is administered, with subsequent, maintenance doses being administered at a lower level.

In some embodiments, the enriched population of genetically engineered CD4/CD8 T cells having the CD45RA^{int}CD45RO^{int} phenotype are locally administered to a subject to improve T cell trafficking to the targeted site, such as a solid tumor site of the subject. In some embodiments, local administration to a tumor cite can include intratumoral, intracranial, intrapleural and hepatic artery delivery.

In some embodiments, genetically engineered CD4/CD8 T cells having the CD45RA^{int}CD45RO^{int} phenotype can be loaded on or in a biopolymer device allowing for T cell proliferation. The T cell loaded device can then be implanted directly to a targeted site in a subject in order to improve trafficking and tumor infiltration.

The dose, e.g., number of the genetically engineered CD4/CD8 T cells having the CD45RA^{int}CD45RO^{int} phenotype administered should be sufficient to effect, e.g., a therapeutic or prophylactic response, in the subject or animal over a reasonable time frame. For example, the number of the genetically engineered CD4/CD8 T cells having the CD45RA^{int}CD45RO^{int} phenotype should be sufficient to bind to a cancer antigen, or treat or prevent cancer in a period of from about 2 hours or longer, e.g., 12 to 24 or more hours, from the time of administration. In certain embodiments, the time period could be even longer. The number of the genetically engineered CD4/CD8 T cells having the CD45RA^{int}CD45RO^{int} phenotype will be determined by, e.g., the efficacy of the genetically engineered CD4/CD8 T cells having the CD45RA^{int}CD45RO^{int} phenotype and the condition of the animal (e.g., human), as well as the body weight of the animal (e.g., human) to be treated.

The number of the of genetically engineered CD4/CD8 T cells having a CD45RA^{int}CD45RO^{int} phenotype also will be determined by the existence, nature and extent of any adverse side effects that might accompany the administration of an enriched population of genetically engineered CD4/CD8 T cells having the CD45RA^{int}CD45RO^{int} phenotype. Typically, the attending physician will decide the number of the inventive genetically engineered CD4/CD8 T cells having the CD45RA^{int}CD45RO^{int} phenotype with which to treat each individual patient, taking into consideration a variety of factors, such as age, body weight, general health, diet, sex, route of administration, and the severity of the condition being treated. By way of example and not intending to limit the invention, the number of the genetically engineered CD4/CD8 T cells having the CD45RA^{int}CD45RO^{int} phenotype can be about 10×10⁴ to about 10×10¹¹ cells per infusion, about 10×10⁵ cells to about 10×10⁹ cells per infusion, or 10×10⁷ to about 10×10⁹ cells per infusion. The inventive genetically engineered T cells may, advantageously, make it possible to effectively treat or prevent cancer or an infectious disease by administering about 100 to about 10,000-fold lower numbers of cells as compared to adoptive immunotherapy protocols that do not administer genetically engineered CD4/CD8 T cells having the CD45RA^{int}CD45RO^{int} phenotype.

For purposes of the inventive methods, the administered genetically engineered CD4/CD8 T cells having the CD45RA^{int}CD45RO^{int} phenotype can be cells that are allogeneic or autologous to the host or subject. Preferably, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the provided therapeutic methods include administration of two or more different engineered T cells, e.g., in the same composition and/or in separate compositions, respectively containing the two or more engineered T cells, each of which specifically recognizes or binds to a first and second, and optionally third, and so forth, antigens.

It is contemplated that the genetically engineered CD4/CD8 T cells having the CD45RA^{int}CD45RO^{int} phenotype can be used in methods of treating or preventing cancer. In this regard, a method of treating or preventing cancer in a mammal can include administering to the subject any of the pharmaceutical compositions including genetically engineered CD4/CD8 T cells having the CD45RA^{int}CD45RO^{int} phenotype described herein in an amount effective to treat or prevent cancer in the mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia (CLL), hairy cell leukemia, acute lymphocytic leukemia (ALL), acute myeloid leukemia (AML), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

In some embodiments, a composition comprising the genetically engineered CD4/CD8 T cells having the CD45RA^{int}CD45RO^{int} phenotype can be administered in combination with an agent that increases the anti-cancer effects of the composition. The genetically engineered CD4/CD8 T cells having the CD45RA^{int}CD45RO^{int} phenotype may be co-administered to a subject with any cancer treatment known in the art.

In one embodiment, the subject is treated with genetically engineered CD4/CD8 T cells having the CD45RA^{int}CD45RO^{int} phenotype and an antiproliferative agent. Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

In one embodiment, the subject is treated with genetically engineered CD4/CD8 T cells having the CD45RA^{int}CD45RO^{int} phenotype and a chemotherapeutic agent. Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In one embodiment, the subject is treated with genetically engineered CD4/CD8 T cells having the CD45RA^{int}CD45RO^{int} phenotype and another anti-tumor agent, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine. Anti-angiogenic agents are well known to those of skill in the art. Suitable anti-angiogenic agents for use in the methods and reprogrammed T cells of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In some embodiments, the disease or condition is an infectious disease or condition, such as, but not limited to, viral, retroviral, bacterial, and protozoan infections, immunodeficiency, Cytomegalovirus (CMV), Epstein-Barr virus (EBY), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

Once the cells are administered to a mammal (e.g., a human), the biological activity of the engineered cell populations in some embodiments is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNy, IL-2, and TNF. In some aspects, the biological activity is measured by assessing clinical outcome, such as reduction in tumor burden or load.

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### EXAMPLE

### Phenotyping Long-Lived RAintROint CAR T Cells

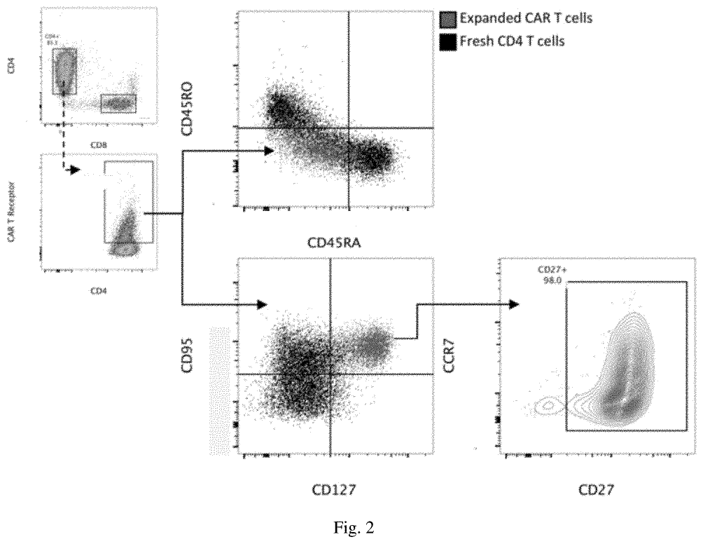

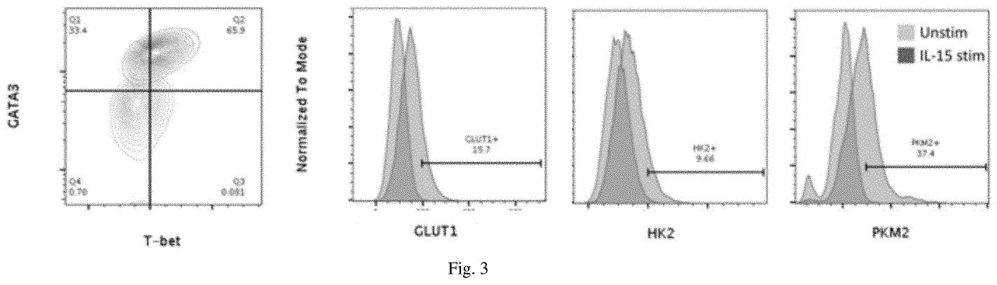

In recent years, immunotherapy using Chimeric Antigen Receptor (CAR) T cells against tumor specific antigens has proven beneficial for cancer treatment and tumor eradication. Patient-to-patient variability, driven by changes in tumor microenvironment and intrinsic diversity of personalized CAR T cells, has been a key reason for incomplete efficacy of this highly promising therapy. We have identified a subset of CD4 and CD8 CAR T cells that have phenotypic and molecular attributes of long-lived pluripotent stem T cells. This subset is primarily characterized by intermediate co-expression of CD45RA and CD45RO (RAintROint). This RAintROint population homogenously expresses CD95 (Fas; a marker that distinguishes Tscm from naïve cells), CD127 (IL7R; essential for maintaining homeostatic proliferation) and CD27 (marker of central memory T cells) (FIG. 2). In line with a stem central memory T cell (Tscm) like phenotype, protein levels of key T helper 1 and T helper 2 transcription factors (T-bet and GATA-3) are absent in this subset (FIG. 3), confirming the lack of commitment of this cell subset. Furthermore, glycolytic enzymes (typically associated with an effector T cell phenotype) are down-regulated in these cells (FIG. 3).

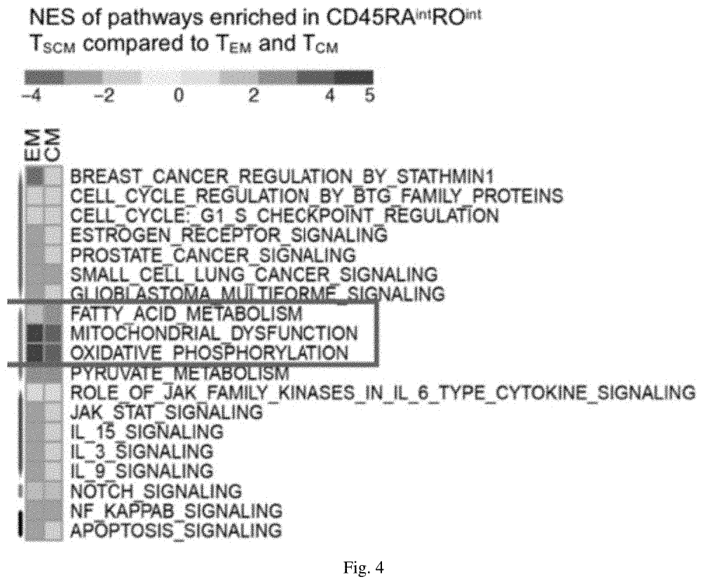

We have previously observed this phenotype in total CD4 T cells, but was never specifically attributed to CAR T cells. This uncommitted phenotype was supported by a gene-expression prolife akin to a quiescent phenotype (upregulation of fatty acid metabolism and oxidative phosphorylation, and downregulation of cell cycling pathways) (FIG. 4). Finally, and most remarkably, these cells retain the capacity to differentiate into all T cell subsets, including the effector subset. These data imply that the RA^{int}RO^{int} subset is a long-lived T cell subset that is capable of self-renewing and re-populating effector compartments. Such unique cell type can prove to be crucial for effective, long-lasting immune responses against the tumor. Below, we have devised a platform for the development of a long-lived and pluripotent CAR T population, with a capacity of effector differentiation.

Effector Differentiation, Enrichment and Self-Renewal of the RA^{int}RO^{int} Subset of CAR T Cells

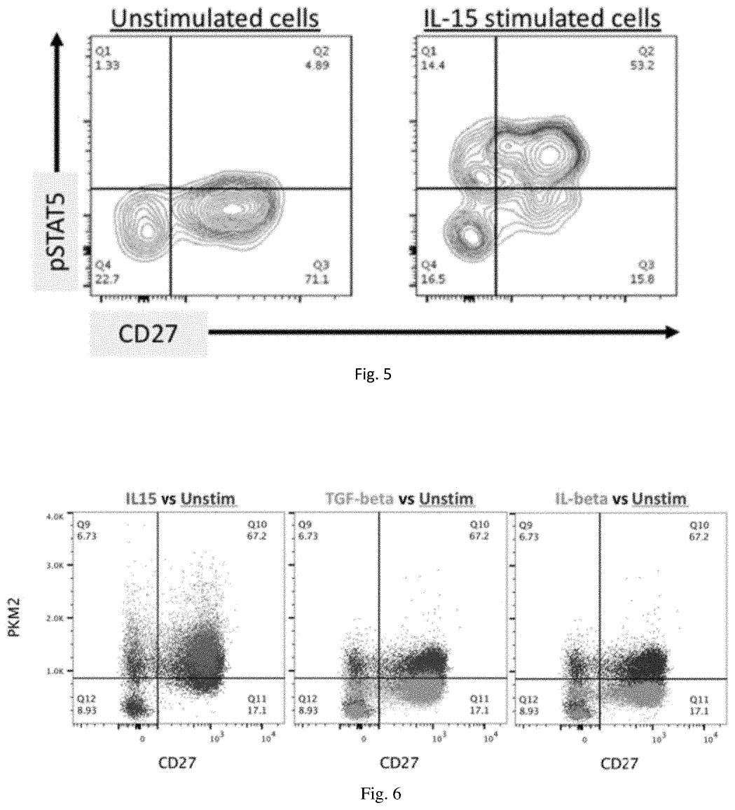

As described above, the RA^{int}RO^{int} CAR T population shows an uncommitted differentiation program which is highlighted by reduced glycolytic activity. We tested the change of lineage commitment within these cells upon the addition of an effector cytokine, IL-15. We observed that IL-15 stimulated CAR T cells swiftly upregulated phospho-STAT5 (a transcription factor directly regulated by IL-15 signal transduction) and demonstrated a shift towards the effector phenotype by downregulating CD27 (FIG. 5). In addition, RA^{int}RO^{int} CAR T cells exposed to IL-15 had heightened metabolic activity and increased protein levels of the master transcription factors GATA-3 and T-bet (FIG. 3). Together, these results support the possibility that an effector phenotype deriving from the RA^{int}RO^{int} population can be induced by IL-15 and other compounds which we will be screening in the near future.

Unlike stimulation with IL-15, addition of TGFβ and IL-1β led to the maintenance of the RA^{int}RO^{int} phenotype. In addition to the maintenance of the uncommitted differentiation status of RA^{int}RO^{int} cells, TGF-β and IL-1β downregulated the glycolytic machinery below the baseline levels (FIG. 6). The role of TGF-β as a sustainer of hematopoietic stem cell phenotype has previously been reported. The novelty of our findings highlights the role of these two cytokines and possibly others, in the maintenance of long-lived pluripotent CAR T cells.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

Having described the invention, the following is claimed:

1. A method of generating an enriched population of CD4/CD8 T cells; the method comprising:
isolating T cells from a biological sample of a subject;
separating a population of CD4/CD8 T cells having a CD45RA^{int}CD45RO^{int} phenotype from the isolated T cells; and
genetically modifying the isolated T cells to express single or multiple antigen-specific receptors.

2. The method of claim 1, wherein the biological sample comprises isolated peripheral blood mononuclear cells from the subject.

3. The method of claim 1, wherein the isolated T-cells are CD4+ T cells.

4. The method of claim 1, wherein the isolated T-cells are CD8+ T cells.

5. The method of claim 1, wherein the separated CD4/CD8 T cells express at least one of CD95, CD127, or CD27.

6. The method of claim 1, wherein the separated CD4/CD8 T cells intermediately express 4-1BB.

7. The method of claim 1, wherein the separated CD4/CD8 T cells express at least one of IL17RA, CD5, IL2RG, IGF2R, SLC38A1, IL7R, SLC44A2, SLC2A3, CD96, CD44, CD6, CCR4, IL4R, or SLC12A7.

8. The method of claim 1, wherein the separated CD4/CD8 T cells have a CD45RA^{int}CD45RO^{int}CD95+CD127+CD27+phenotype.

9. The method of claim 1, wherein the separated CD4/CD8 T cells have a CD45RA^{int}CD45RO^{int}CD95+CD127+CD27+IL7R+CD44+SCL38A1+IL2RG+CD6+CD5+phenotype.

10. The method of claim 1, further comprising activating the isolated CD4/CD8 T cells with an anti-CD3 antibody and/or an anti-CD28 antibody.

11. The method of claim 1, further comprising culturing the isolated CD4/CD8 T cells in an amount of IL7 and IL15 effective to promote expansion and/or formation of an enriched population of CD4/CD8 T cells having a CD45RA^{int}CD45RO^{int} phenotype.

12. The method of claim 1, further comprising culturing the separated CD4/CD8 T cells having a CD45RA^{int}CD45RO^{int} phenotype in a culture medium comprising TGFβ/IL1β to maintain the CD45RA^{int}CD45RO^{int} phenotype.

13. The method of claim 1, wherein the antigen-specific receptors are chimeric antigen receptors (CARs).

14. The method of claim 1, wherein the antigen-specific receptors recognize a cancer related antigen.

15. The method of claim 1, wherein the isolated T cells are genetically modified by at least one of transduction, transfection, and/or electroporation.

16. The method of claim 13, wherein the antigen-specific receptors include an extracellular antigen binding domain that targets an antigen comprising at least one of CD19, CD20, CD22, ROR1, TSLPR, mesothelin, CD33, CD38, CD123 (IL3RA), CD138, BCMA (CD269), GPC2, GPC3, FGFR4, c-Met, PSMA, Glycolipid F77, EGFRvIII, GD-2, NY-ESO-1 TCR, or MAGE A3 TCR.

17. The method of claim 1, wherein the T cells are T cells of a human having cancer.

18. A method of treating cancer in a subject in need thereof, the method comprising:
administering to the subject an enriched population of genetically modified antigen-specific receptor T cells produced by a method of claim 1, wherein at least 50% of T-cells of the population have a CD4+CD8+CD45RA^{int}CD45RO^{int} phenotype.

19. The method of claim 18, wherein the T-cell population upon administration to a subject with cancer is capable of promoting in vivo expansion, persistence of patient specific anti-cancer T-cells resulting in cancer reduction, elimination, and/or remission.

20. The method of claim 18, wherein the cancer is a hematological cancer, and wherein the hematological cancer is leukemia, lymphoma, or multiple myeloma.
